(19) United States
(12) Patent Application Publication
(10) Pub. No.: US 2023/0210539 A1
(43) Pub. Date: Jul. 6, 2023

(54) ELASTIN FORMATION USING FIBROUS IMPLANTS

(71) Applicant: STENTIT BV, Eindhoven (NL)

(72) Inventors: Maria SOL CABRERA, Eindhoven (NL); Bart SANDERS, Eindhoven (NL)

(21) Appl. No.: 17/998,922

(22) PCT Filed: May 14, 2021
(86) PCT No.: PCT/EP2021/062891

§ 371 Date: Nov. 15, 2022

(30) Foreign Application Priority Data
May 15, 2020 (NL) 2025593

(51) Int. Cl. A61B 17/12; A61F 2/24; A61F 2/90; A61M 27/00; A61F 2/07

(52) U.S. Cl. CPC A61B 17/12177; A61F 2/2412; A61F 2/90; A61M 27/002; A61F 2/07; A61B 2017/00893

(57) ABSTRACT

A cardiovascular fibrous implant for rebuilding elastin and the use of such an implant, wherein the implant is comprised of fibers forming a network, and wherein the fibers comprised in said network have a fiber diameter of 150 μm or less.

### TECHNICAL FIELD

The present disclosure relates to the field of regenerative medicine, more particularly in situ tissue engineering, using fibrous implants with the aim to rebuild elastin.

### BACKGROUND

### Elastin

Elastin is a key extracellular matrix (ECM) component that provides resilience and elasticity. Elastin can be found in many dynamic tissues and organs such as lungs, skin and ligaments, but also in cardiovascular tissues such as blood vessels and heart valves. Besides providing a mechanical function, elastin also has a biological protective function, regulating the cellular response via biomechanical transduction to maintain homeostasis.

Elastin is a protein composed of a cross-linked monomer called tropoelastin that can be produced by synthetic cells such as fibroblasts, endothelial cells (ECs), chondrocytes and smooth muscle cells (SMCs). Elastin is produced predominantly during late fetal, neonatal and early postnatal growth, and stagnates almost fully after puberty. The formation of elastin is also better known as elastogenesis.

In healthy tissues elastin degrades slowly, having an approximate half-life of 70 years. However, both genetic and acquired cardiovascular diseases such as atherosclerosis, aneurysm formation, vascular stiffening and calcifications, are associated with a progressive loss of elastin or elasticity that could compromise vital organs resulting in hypertension, stroke, ischemia, internal bleedings, or valve failure.

The development of cardiovascular implants that can incorporate elastin or stimulate its biosynthesis becomes an interesting approach to secure integrity and functionality of these substitutes.

### Smooth Muscle Cells (SMC)

SMCs are specialized contractile cells present in organs such as blood vessels, the gastrointestinal tract and the respiratory system. Under physiological conditions, SMCs possess a quiescent contractile phenotype that controls constriction and dilation of blood vessels. However, under pathological conditions they convert into a non-contractile and synthetic phenotype that results in cell proliferation and increased ECM production.

In native blood vessels, the SMCs are embedded in between the elastic lamellae or fibers. The protective role of this structure is important to regulate the response of SMCs, and to control their phenotypic modulation, proliferation, and migration. The production of ECM components depends on the signals passed through elastin. Damage of elastic fibers could therefore result in migration and proliferation of SMCs to the innermost layer of the vessel, leading to vascular narrowing. In order to prevent this SMC response, immunosuppressive drugs can be administered to act on the metabolism, growth, and proliferation of the cells to suppress neointimal hyperplasia. Nevertheless, the temporary effect of drugs might result in late-occlusion.

Techniques for overcoming elastin damage are to date nonexistent. Since after youth, elastin is no longer naturally synthesized by the human body, the development of cardiovascular implants that can incorporate elastin or trigger SMCs to stimulate elastin biosynthesis, becomes an interesting approach to prevent intimal hyperplasia and has promising long-term outcomes. Here, restoring elastin could induce a phenotypic switch in which SMCs get from a proliferative and non-contractile state in the absence of elastin, to a quiescent and contractile state in the presence of elastin (FIG. 1).

### Surgical Implants

For the treatment of cardiovascular diseases, current surgical interventions require the use of grafts to bypass obstructed vessels. Due to their native nature, the preferred grafts are blood vessels extracted from elsewhere in the patient's body or from another donor. Long-term patency rates of such autologous implants are also associated with elastin content. It is known that grafts made out of the left internal mammary artery (LIMA), which is rich in elastin content, have superior clinical outcomes compared to the superficial femoral artery (SFA) which has a lower elastin content. When such biological grafts are not available, synthetic grafts made from Teflon and Dacron, for example, can be used. Nevertheless, the long-term functionality of synthetic implants is not that straight forward as re-occlusion remains a common failure point, especially for small diameter applications.

### Endovascular Implants

Minimally-invasive techniques to treat obstructive vascular diseases, on the other hand, involve the use of balloons and stents. These techniques are an effective way to instantly relieve the obstruction and to restore the passage of blood without a surgical procedure. However, their use might lead to vascular tissue damage and SMC activation resulting in in-stent restenosis. Therefore, drugs are incorporated into stents and balloons to inhibit SMC activation. Nevertheless, the attenuating effect of drugs cannot solve the underlying damage of the elastic fibers and still could result in late in-stent restenosis.

### In-Vitro Tissue Engineering

Tissue engineered blood vessels are an attractive alternative to design responsive, living conduits with properties that are similar to native tissues. This would allow the provision of viability, availability, and compatibility in the absence of native vessels from the patient or a donor. The concept consists of using three-dimensional biological or synthetic scaffolds to support cell adhesion, migration, differentiation and proliferation in addition to guidance for neo tissue formation.

The activation of synthetic cells is key to rebuilding essential ECM components. It is known that SMCs cultured in conventional tissue culture dishes can secrete elastin. Static stretching of SMCs stimulates both elastin formation and crosslinking. Furthermore, the exposure of SMCs to a certain level of shear stress can determine whether or not elastin is formed. Therefore, the creation of elastin in a three-dimensional tubular structure in-vitro could be pursued provided that the scaffold can secure the right chemistry and stimuli for seeded SMCs to secrete elastin. This approach could provide the possibility of securing the presence of key tissue components in cultured vessels that could be later surgically implanted in the patient as a bypass or interposition.

### In Situ Tissue Engineering

In situ tissue engineered vessels aim to use the regenerative potential of the human body to recruit endogenous cells into the scaffolds in-vivo. This exposes the scaffold to a cascade of inflammatory events, where immune cells such as monocytes and macrophages can steer cell response and neo tissue formation. Polarization of macrophages towards a pro-inflammatory M1 type, which encapsulates the implant and results in chronic inflammation, or pro-healing M2 type, which resorbs the implant and allows for SMC activation leading to synthesis or degradation of ECM components, depends once again on the structure of the scaffold. Therefore, the formation of elastin in a three-dimensional tubular structure in-vivo could be pursued provided that: 1) synthetic cells from the patient such as SMCs can reach the scaffold, and 2) the scaffold can secure the right chemistry and stimuli for synthetic cells to secrete sufficient elastin. This approach could provide the possibility of gradually replacing a surgically implanted scaffold by a new vessel created by the patient's own cells.

To date, minimally-invasive techniques have been aimed at relieving obstructions without removing the native artery and at surgical techniques aimed at replacing or bypassing segments of the artery. The combination of these available and under development concepts such as scaffolds and grafts mounted on stents, might enable new possibilities and allow the pursuit of a minimally-invasive implantation of vascular prosthesis. However, the multiple factors affecting these techniques might result in suboptimal treatments. The use of conventional stents affects, temporarily or permanently, the elastic properties of the arteries, yielding undesired consequences in terms of functionality and long-term patency of the implanted segment. Further, the use of drugs to attenuate the proliferative response after stent placement might inhibit the reconstructive capacity of in situ scaffolds.

The present invention advances the art by disclosing a fibrous implant for cardiovascular applications in which host cells interact with the fibrous implant to trigger the synthesis of elastin using an in situ tissue engineering approach.

### SUMMARY

The present invention provides a fibrous implant for cardiovascular applications that allows the rebuilding of valuable ECM components such as elastin. The fibrous implant is composed of fibers having a diameter in the micrometric and nanometric range. These fibers form a network of stacked fibers, that allows host cells to adhere and synthesize elastin.

In one variation, the pore size of the fibrous network can be controlled to enable or prevent host cell infiltration and/or to steer cell signaling. In yet another variation, the fibrous implant is made out of a bioabsorbable polymer. In still another variation, the fibrous implant can comprise at least partially a drug. In still another variation the fibrous implant is pre-stretched and/or maintains residual stress. In still another variation, the morphology of the network either comprises a random or aligned fiber organization, or a combination thereof. In still another variation, the stiffness of the fibrous implant has been optimized to enable elastin formation. In still another variation, the fibrous implant is exposed to dynamic mechanical stimulation.

Further disclosed is the distinct positioning of the implant with respect to the host tissue, in which in one embodiment at least a part of the fibrous implant is brought in direct contact with the host tissue, and in which the host tissue could be damaged. In another embodiment, the fibrous implant is applied to recipients where the immune response is in homeostasis. Yet in another example, the embodiment is in direct contact with the blood flow being exposed to shear stress. Furthermore, an embodiment describes that fibrous implants can control osmotic pressures and gradients.

Also disclosed are methods in which certain embodiments can be used to either partially replace or repair an existing structure, where other embodiments can be used to create new structures or completely replace existing structures. In some embodiments, the fibrous implant is a tubular conduit that can act either as a stent, scaffold, graft, or shunt and can be positioned intraluminally, or as an interposition, in which endoluminal elastin can be formed. In another embodiment, fibrous tubular implants can contain valves or meshes to provide additional functionality or act as embolization or occluding devices. In yet another embodiment, the fibrous implant is a patch to restore elastin inside or on top of a body part.

In addition, clinical applications for the use these fibrous implants are disclosed. Some embodiments describe the use for endovascular applications such as balloon angioplasty, atherectomy, subintimal bypassing, aneurysm repair, or as an occluding device. Yet another embodiment describes the use for surgical applications. One embodiment discloses the use of fibrous implants to repair endoluminal elastin to inhibit disease progression such as neointimal hyperplasia and/or atherosclerosis. Another embodiment makes use of the reconstruction of a media layer with elastin to take over the hemodynamic load, prevent bursting of aneurysmatic vessels and restore vascular compliance. In yet another embodiment, elastin can be restored in ventricle or atrial septum defects to ease compliance to septum deformation, restore the heart wall, repair valve leaflets, or provide external reinforcement of an aneurysm.

Even more, methods are described disclosing the production of elastin-forming implants and the possibility of combining them with additional support of delivery devices. Other embodiments pertain to a method of producing an elastin-forming fibrous network directly onto and/or into the target body part. Also provided herein are ways of testing and evaluating the elastin-forming capacity of fibrous implants.

### BRIEF DESCRIPTION OF THE FIGURES

FIG. 1: Pictoral representation of how elastin interaction with SMCs could switch cell phenotype from a proliferative and non-contractile state in the absence of elastin, towards a quiescent and contractile state in the presence of elastin.

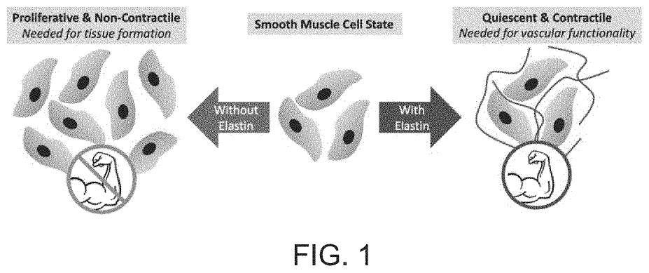

FIG. 2: Example of the network of a fibrous implant that can induce elastin formation.

FIGS. 3A-D: Example of a fibrous implant placed inside the lumen of a peripheral artery of a rabbit, showing endoluminal elastin formation after 3 months of implantation.

FIGS. 4A-B: Example of two fibrous implants placed inside the lumen of a peripheral artery of a rabbit in which the upper bi-layered implant (4A) did not show predominant elastin formation after 3 months, but the lower uni-layered implant (4B) did.

FIGS. 5A-B: Data from endoluminal fibrous implants in which luminal smooth muscle cell migration colocalized with luminal elastin formation.

### DETAILED DESCRIPTION

Unless otherwise defined herein, scientific and technical terms used in connection with the present invention shall have the meanings that are commonly understood by those of ordinary skill in the art. Further, unless otherwise required by context, singular terms shall include the plural and plural terms shall include the singular. The methods and techniques disclosed are generally performed according to conventional methods, well known in the art and as described in various general and more specific references that are discussed throughout the present specification unless otherwise indicated.

The term “elastin” as used herein, relates to the ECM component elastin. It refers to both its precursor form as tropoelastin as well as its mature form as a protein formed by the cross-linking of tropoelastin. It also includes elastic fibers, as well as their arrangement as a network forming fenestrae sheets or lamella.

The terms “regenerate”, “restore”, “reconstruct”, “rebuild”, “renew”, and “repair” as used herein, relate to the ability to form new biological components. In the context of elastin, it refers to the ability to create new elastin.

The term “synthetic cell” as used herein, refers to biological cells that have the ability to synthesize proteins, ECM components, or tissue. These can be, for instance, fibroblasts, fibrocytes, SMCs, ECs, mesenchymal stem cells, and immune cells such as, but not limited to, monocytes and macrophages.

The terms “bioresorbable”, “bioabsorbable”, “biodegradable”, and “biodegradation” as used herein, relate to the ability of a material to disintegrate within the body and be cleared from the body.

The term “tubular” as used herein, is of or pertaining to an approximate shape of a cylinder.

The “intimal layer” (or tunica intima or intima), refers to the innermost layer in an unmodified, healthy blood vessel. It is made up of one layer of ECs and is supported by an internal elastic lamina, in which the Ecs are in direct contact with the blood flow.

The “medial layer” (or tunica media, or media), refers to the middle layer of a blood vessel. It is made up of SMCs and elastic tissue, and it lies between the tunica intima on the inside and the tunica externa on the outside.

The terms “recipient”, “patient”, “host”, “subject” are used interchangeably herein and include, but are not limited to, an organism; a mammal, including e.g., a human, non-human primate, mouse, pig, cow, goat, cat, rabbit, rat, guinea pig, mini pig, hamster, horse, monkey, sheep or other non-human mammal; and a non-mammal, including, e.g., a non-mammal vertebrate, such as a bird (e.g., a chicken or duck), an amphibian and a fish, and a non-mammalian invertebrate.

The invention describes a bioresorbable fibrous implant for cardiovascular applications with the capacity to reconstruct elastin. The implant is composed of micrometric and/or nanometric fibers, forming a three-dimensional fibrous network. The fibers can be spaced creating multiple interconnected pores throughout the perimeter and thickness of the network providing host cells with the ability to adhere to the fibers and migrate over and/or into the network. Such host cells can be immune cells responding to the implant. Because of the micrometric and/or nanometric scale of the fibers in the network, the immune cells can be triggered to initiate a favorable pro-healing immune response. Synthetic cells that adhere to the fibrous implant can be triggered to synthesize new biological components into and/or onto the implant including elastin. In order to trigger the host cells to form elastin, controlling the characteristics of the fibrous implant is fundamental. The parameters that define the characteristics influencing elastogenesis are described in further detail below.

### Methods of Making

In-situ tissue engineering of elastin by fibrous implants can be triggered by a number of attributes, as further disclosed below, which may act individually or in combination thereof in favor of inducing elastin formation.

### Fibers

Fiber size can affect both immune cells as well as tissue producing cells Immune cells in relation to smaller fibers, could initiate a pro-healing (M2-type) response, where larger sized fibers could result in a more pro-inflammatory (M1-type) response. The onset of either a pro-healing or pro-inflammatory response can have an indirect effect on the behavior of synthetic cells to either induce a regenerative response in which elastin could be restored, or chronic inflammatory response in which the implant undergoes fibrotic encapsulation.

Regardless of the initiated immune response, the fiber size could also affect the behavior of synthetic cells that are in direct contact, which could switch to a regenerative or fibrotic response depending on the fiber size.

Depending on the production settings and materials used, one embodiment has fibers with a fiber diameter ranging between 1 nanometer (nm) to 500 micrometer (μm) and anything in between. To enable a pro-healing response that promotes elastin formation, fibers can have a diameter of 500 μm or less, 250 μm or less, 150 μm or less, 100 μm or less, 50 μm or less, 25 μm or less 15 μm or less, 10 μm or less, 9 μm or less, 8 μm or less, 7 μm or less, 6 μm or less, 5 μm or less, 4 μm or less, 3 μm or less, 2 μm or less, 1 μm or less, 500 nm or less, 250 nm or less, 150 nm or less, 100 nm or less, 50 nm or less, 25 nm or less 15 nm or less, 10 nm or less, 9 nm or less, 8 nm or less, 7 nm or less, 6 nm or less, 5 nm or less, 4 nm or less, 3 nm or less, 2 nm or less, or 1 nm or less.

In another embodiment, the fibers preferably have a fiber diameter ranges for example between 1 nm and 500 μm, between 1 nm and 250 μm, between 1 nm and 150 μm, between 1 nm and 100 μm, between 1 nm and 50 μm, between 1 nm and 25 μm, between 1 nm and 15 μm, between 2 nm and 15 μm, between 3 nm and 15 μm, between 4 nm and 15 μm, between 5 nm and 15 μm, between 6 nm and 15 μm, between 7 nm and 15 μm, between 8 nm and 15 μm, between 9 nm and 15 μm, between 10 nm and 15 μm, between 15 nm and 15 μm, between 25 nm and 15 μm, between 50 nm and 15 μm, between 100 nm and 15 μm, between 150 nm and 15 μm, between 250 nm and 15 μm, between 500 nm and 15 μm, between 500 nm and 10 μm, between 500 nm and 9 μm, between 500 nm and 8 μm, between 500 nm and 7 μm, between 500 nm and 6 μm, between 500 nm and 5 μm, between 500 nm and 4 μm, between 500 nm and 3 μm, between 1 μm and 3 μm.

### Pores

By tuning the fiber distance and fiber diameter, pore size can be controlled. Depending on the production settings and materials used, pores can be of a mesometric, macrometric, micrometric, or nanometric range or combinations thereof. Pore size can have an effect on cell infiltration as well as on cell signaling, which both can affect elastin formation. In this way, the three-dimensional fibrous tubular network can be filled with cells that can produce biological tissue or tissue components.

### Cell Infiltration

In one embodiment, host cells adhere to the fibers and stay on the surface. In another embodiment, the pore size is sufficiently large for host cells to infiltrate into the network. By controlling the pore size, cell migration through the implant can be either facilitated or inhibited. In this way, cells can produce tissue components, either on top of the fibrous implant, and/or within the fibrous implant. Therefore, the production of tissue components such as elastin, can be controlled in certain parts or locations of the fibrous implant.

To enable cell infiltration that promotes elastin formation, one embodiment has pores with a pore size for example of 1 nm or more, 10 nm or more, 100 nm or more, 150 nm or more, 250 nm or more, 500 nm or more, 750 nm or more, 1 μm or more, 2 μm or more, 3 μm or more, 4 μm or more, 5 μm or more, 6 μm or more, 7 μm or more, 8 μm or more, 9 μm or more, 10 μm or more, 15 μm or more, 25 μm or more, 50 μm or more, 100 μm or more, 150 μm or more, 250 μm or more, 500 μm or more, 1000 μm or more, 1500 μm or more, 2500 μm or more, 5000 μm or more, or 10.000 μm or more.

### Cell Signaling

Pore size can have an effect on cell signaling, triggering synthetic cells to produce elastin. The trigger can be related, for example, to the curvature of such pores or because the pores are sufficiently small enough for cells to span over multiple fibers rather than adhering to an individual fiber. In case of immune cells, similar mechanisms could initiate a pro-healing M2 immune response.

To enable cell signaling that promotes elastin formation, another embodiment has pores with a pore size for example of 10,000 μm or less, 5000 μm or less, 2500 μm or less, 1500 μm or less, 1000 μm or less, 500 μm or less, 250 μm or less, 150 μm or less, 100 μm or less, 50 μm or less, 25 μm or less, 15 μm or less, 10 μm or less, 9 μm or less, 8 μm or less, 7 μm or less, 6 μm or less, 5 μm or less, 4 μm or less, 3 μm or less, 2 μm or less, 1 μm or less, 750 nm or less, 500 nm or less, 250 nm or less, 150 nm or less, 100 nm or less, 10 nm or less, or 1 nm or less.

### Shear Stress

In one embodiment the cells are exposed to shear stress. Shear stress can have a mechanical regulating effect on cells that can trigger elastin formation. Synthetic cells in the vicinity of the blood stream will respond differently than cells located inside the network. The synthetic cells exposed to shear stress from the bloodstream will synthesize elastin, encapsulating themselves into the deposited extracellular elastin. Over time, the extracellular elastin can further mature and merge with the elastin formed from nearby cells, creating an elastin layer onto and/or into the fibrous implant.

The level of shear stress can be controlled by the thickness of the implant. In one exemplary embodiment, increasing the thickness of a tubular fibrous structures can lead to a reduced endoluminal diameter and a higher blood flow velocity that results in higher shear stress. Vice versa, reducing the thickness of the implant can lead to a lower shear stress.

Cells inside the fibrous network are mainly shielded from the blood flow and thus exposed to less shear stress, where cells on the outer surface are exposed to shear stress. In another exemplary embodiment, shear stress is further improved within the network by lowering the density of the fibrous implant, by tuning the pore size, and/or fiber diameter.

In one embodiment, the fibrous implant can have a homogeneous fine-meshed network facing the blood flow, which prevents flow turbulence and instead results in a uniform flow profile that cause lower shear stresses and promotes elastin formation. In this way, cells along the length of the implant exposed to shear stress could form a uniform elastin layer. The transition from a turbulent flow profile toward a uniform flow profile could be controlled by refining the network.

### Materials

The selection of materials from which the fibrous implant is made can have an effect on elastin formation. Degrading objects can initiate an M2 pro-healing response leading to elastin formation in the case where the immune cells are capable of clearing the foreign implant, whereas the response to a permanent implant that cannot be eliminated could lead to a pro-inflammatory M1 response.

### Material Composition

To promote elastin formation, one embodiment is composed of fibers made out of materials including but not limited to:
bioresorbable polymers (such as poly lactic acid (PLA), including poly(L-lactide), poly(D-lactide), poly(D,L-lactide), as well as polyglycolid acid (PGA), polycaprolactone, polydioxanone, poly(trimethylene carbonate), poly(4-hydroxybutyrate), poly(ester amides) (PEA), polyurethanes, poly(trimethylene carbonate), poly(ethylene glycol), poly(vinyl alcohol), polyvinylpyrrolidone, polyhydroxyalkanoate, polyfumuarate, and copolymers thereof),
non bioresorbable polymers (such as polypropylene, polyethylene, polyethylene terephthalate, polytetrafluoroethylene (PTFE), polyaryletherketone, nylon, fluorinated ethylene propylene, polybutester, or copolymers thereof),
biological components (such as hyaluronan, collagen, tropoelastin, elastin, fibrin, gelatin, chitosan, alginate, aloe/pectin, cellulose, or other biological materials originating from tissues from either autologous, allergenic or xenogenic origin),
or a combination thereof.

The polymers can be of the D-isoform, the L-isoform, or a mixture of both. Multiple polymers and copolymers can be mixed and blended into different ratios. The polymer fibers can be crosslinked. Some embodiments may include supramolecular chemistry including supramolecular polymers, linking mechanisms or moieties. Some embodiments may comprise shape-memory polymers.

### Degradation Speed

When the material is biodegradable, the speed of degradation could affect elastin formation. Slow degrading materials could trigger a similar immune response as permanent implants. Fast degrading materials might degrade even before elastin could be formed. While undergoing degradation, the material generally first decreases in averaged molecular weight, followed by a loss in mechanical properties, followed by a loss in polymer mass. As such, one embodiment has an optimized degradation speed to facilitate elastin formation. Another embodiment uses materials with faster or slower biodegradation profiles or a combination thereof. Also, in one variation, the embodiment is partially composed of biodegradable materials, to enable elastin formation, and non-degradable materials.

To promote elastin formation, the implant should remain for a sufficient time in the body. Therefore, in one exemplary embodiment, the fibrous implant loses 50% of its initial averaged molecular weight after, for example, 1 week, 2 weeks, 3 weeks, 4 weeks, 5 weeks, 6 weeks, 7 weeks, 8 weeks, 9 weeks, 10 weeks, 11 weeks, 12 weeks or 4 months, 5 months, or 6 months. In one exemplary variation the embodiment loses 50% of the initial mechanical properties after 1 month, after 2 months, after 3 months, after 6 months, after 9 months, after 12 months, after 18 months. In yet one other exemplary variation, the embodiment undergoes 50% of initial polymer mass loss after 3 months, or after 6 months, or after 9 months, or after 12 months, or after 18 months, or after 24 months, or after 30 months, or after 36 months, or beyond 36 months.

Furthermore, the mechanical properties of the implant might be affected during degradation. Stiff implants might soften in response to mechanical influence. This might enable the possibility of a mechanical transduction effect on the cells leading to elastin production.

### Implantation Site

Placing the fibrous implant into the body can result in local tissue damage by which residing synthetic cells are activated. Hereby, in one exemplary embodiment, synthetic cells migrate from the 1) anastomosis, 2) adjacent tissue, 3) blood stream, or 4) a combination of the options 1, 2, and/or 3, into or onto the fibrous implant. In yet another embodiment different cell types such as endothelial cells, or monocytes and macrophages in contact with the embodiment transdifferentiate into synthetic cells.

### Tissue Damage

The level of tissue damage to promote elastin formation can be controlled and induced prior to, during, or after the implantation procedure. The induction of damage can be a consequence of the intervention or be added induced intentionally in addition to the intervention. The fibrous implant can benefit from the infiltration of such activated synthetic cells, as they can be a source for tissue production and elastin formation. In one embodiment, the fibrous implant is implanted in the vicinity of tissue damage.

Tissue damage could be induced for example by endovascular procedures such as but not limited to: balloon angioplasty, stent placement, atherectomy, laser ablation, intravascular ultrasound, subintimal passage, or other surgical interventions such as an anastomosis, shunt, bypass or MIDCAB procedure.

### Drugs

In one embodiment, drugs are incorporated to steer elastin formation. In this way, synthetic cells can be influenced by affecting, for example, their metabolism, growth, proliferation, migration.

In another embodiment, drugs are incorporated with concentration gradients throughout the network, or on a particular surface. In yet another embodiment drugs are incorporated inside the fiber, and/or coating the fiber. In another embodiment, drugs are incorporated by filling the pores of the network with a drug containing filler, for example a drug loaded hydrogel.

Drugs can have different functions, acting as immunosupressive, anti-proliferative, migration inhibitors, anti-inflammatory, anti-thrombotic and/or prohealing agents. Depending on the desired effect, drugs or a combination of drugs, can be incorporated to prevent cell migration, cell proliferation, or cell differentiation, and to limit the inflammatory response upon damage to the vascular wall, or to stimulate vascular healing. Drugs can act on virtually any cell or tissue type, for example synthetic cell types such as SMCs and fibroblasts, but also endothelial or immune cells.

Examples of immunosuppressive drugs include sirolimus, tacrolimus, everolimus, zotarolimus, M-prednisolone, dexamethasone, cyclosporine, mycophenolic acid, mizoribine, interfereon-1b, tranilast, leflunomide, myolimus, novolimus, pimecrolimus and biolimus A9.

Examples of anti-proliferative drugs include taxol (paclitaxel), actinomycin, methotrexate, angiopeptin, vincristine, mitomycin, statins, c-myc antisense, abbott ABT-578, RestenASE, 2-choloro-deoxyadenosine, BCP678, Taxol derivative (QP-2) and PCNA ribozyme.

Examples of migration inhibitors include batimastat, prolylhydrosylase inhibitors, halofunginone, C-proteinase inhibitors, Probucol and Metalloproteinase inhibitors.

Examples of drugs to enhance healing include BCP671, VEGF, 17-beta-estradiol, NO donor compound, EPC antibodies, TK-ase inhibition, HMG CoA reductase inhibitor, Biorest, MCP-1, SDF-1-alpha, IL-4, IL-8, MIP-1beta, TGF-beta, bFGF, PDGF, MMPs, TIMPs, IL-12, IL-6, IL-1beta, TNF-alpha, IL-13, IL-4, IL-10, PGE₂ and IFN-gamma.

Examples of anti-thrombins include heparin, hirudin and iloprost, abciximab.

Drugs can also be moieties, proteins, enzymes, peptides, molecules or atoms.

### Immune Response

The immune cells that respond to the fibrous implant can trigger a pro-healing response which is favorable for elastin formation. However, it can occur that the patient receiving the implant has a comorbidity such as diabetes, atherosclerosis, or other local or systemic disease. Such disease environment could have an effect on the immune system, which might result in a skewed pro-inflammatory response.

In order to create a local pro-healing response, one embodiment could be incorporated with pro-healing immunomodulating drugs.

### Age Dependency

Age can play a role in elastin formation. The fibrous implant in contact with either a young or old person might affect elastin formation. Younger children have a less developed adaptive immune response, which matures over time. This might explain why native elastin formation stops by the end of puberty, as there might be a relation with the maturity of the adaptive immune response.

Furthermore, it should not be neglected that aging by itself is affecting the conditions of the vasculature. Besides, patients might have underlying comorbidities such as diabetes or atherosclerosis which might interfere with the formation of elastin.

In one embodiment, elastin is formed in selective recipients grouped by age and/or co-morbidities

### Mechanical Factors

### Pre-Stress & Residual Stress

Pre-stretch and residual stress have a direct or indirect influence on the biomechanical behavior of many biological tissues. Pre-stretch can be described as geometrical changes while maintaining the volume. Upon release of in vivo boundary conditions, pre-stretch leads to tissue retraction. Residual stress, on the other hand, can be defined as the stress that remains in the tissue after its original cause has been removed. Upon release of boundary conditions, residual stress leads to an artery ring springing open into a sector after a radial cut. It is known that stretching can stimulate the synthesis of matrix components via SMCs in vitro. It is also known that residual stresses are crucial to maintain homeostasis in arteries and is closely related with the elastin expression. Therefore, one embodiment is pre-stretched in the body to induce residual stresses and promote elastin formation

Fibers can have different spatial arrangements within the network. The network can be composed of straight fibers that can be parallel to each other or forming an angle with respect to other fibers, waved fibers, fibers forming loops or a combination thereof. These fiber arrangements can be altered by a mechanical stimuli acting on the implant, such as internal pressure in a tubular construct, or when being exposed to (cyclic) stretch in a dynamic location. The initial arrangement of the network prior to mechanical loading defines the resulting level of pre-stretch in the implant. Networks with mainly straight fibers will experience higher levels of pre-stretch compared to wavy or even looped meshes which first have to undergo uncoiling of the loops before entering the stretch phase. In this way, the level of pre-stretch in the network of fibrous implants can be controlled by tuning the spatial arrangement of the individual fibers forming the network, affecting the degree of waviness of the individual fibers or the angle between straight fibers. Therefore, prior to mechanical loading conditions, one embodiment is composed of at least partially straight fibers, where another embodiment is composed of at least partially waved fibers, where yet another embodiment is composed of at least partially looped fibers, or where yet another embodiment is composed of a combination of at least partially straight, waived, or looped fibers.

### Substrate Stiffness

The stiffness of the substrates to which tissue producing cells adhere to, is known to regulate the proliferation, migration, and differentiation of cell types, but also the composition and amount of ECM being produced. Also, immune cells such as macrophages and monocytes respond to substrate stiffness, by which a careful material selection of the implant might steer the initiated immune response.

In one embodiment, the stiffness of the substrate has been optimized for elastin formation. Substrate stiffness can be controlled by selecting the materials from which the fibers are made. The thickness of the fibers can be increased to affect local fiber stiffness. Other possibilities might be to cross-link the networks to enhance the stiffness of the network in general. Other ways are to increase the density of the network, or make the constructs thicker, by which they are less responsive to mechanical influence.

Substrate stiffness can also be affected by implant degradation. In case the fibrous implant is made from a biodegradable material, bioresorption will result in a loss in mechanical properties over time, which can alter substrate stiffness.

### Dynamic Stimulation

Certain biological locations are prone to dynamic loading conditions. Implants in the blood stream for instance, can be subjected to pulsatile flow by which the interaction of the artery with the endovascular implant creates a dynamic loading condition. Switching from a constant to a dynamic loading condition either in stress and/or strain could affect the response of tissue producing cells and immune cells which might affect elastin formation. Therefore, one embodiment is exposed to dynamic mechanical stimulation to induce elastin formation.

### Network Morphology

The fiber network organization can be controlled such that more randomly organized networks are created or more controlled (aligned) networks are formed. The morphology of such network may have a downstream effect on the elastin formation, wherein the formation of elastin might be triggered or not in either a random configuration or a controlled configuration or a mixture thereof.

Even so, the fibers of the network can result in contact guidance of the cells, in which cells also align as such following the organization of the fibrous network. In the case of a more controlled network with circumferential organization, circumferential elastin formation could be achieved as such. Therefore, one embodiment might be composed of either random and/or controlled fibers.

### Osmotic Pressure

Density of the fibrous network could affect the exchange of molecules, proteins, and cytokines by which osmotic gradients could occur. Especially once cells start filling the pores with tissue, a dense structure can be obtained. This could lead to differences in osmotic gradients over and/or within the fibrous implant leading to cell migration. In this way, one embodiment comprises at least one layer where the density of the network, either with or without tissue production, could facilitate cellular interaction to steer elastin formation.

### Methods of Applying

In one embodiment, the implant is used for repairing or replacing cardiovascular tissue, organs, or parts thereof. By using fibrous implants for cardiovascular application, those devices can be used to restore damaged or diseased biological tissues in need of elastin repair. Hereby, disease progression could be slowed down, stopped, or even reverted, and damaged tissues could be repaired to prevent associated complications. In the field of cardiovascular applications, restoring elastin is of particular interest for the application of vessels and valves. For cardiovascular applications they could be for instance in arteries, veins, capillaries, arterioles, or lymphatic vessels, or heart valves such the pulmonary, aortic, tricuspid and mitral valve, and also venous valves and lymphatic valves.

### Implantation Procedure

In one embodiment, the fibrous implant can be used to repair or replace existing structures, or create new structures. In one example, the existing structure is first removed, and then the fibrous implant is placed. In another example, the existing structure at least partially is first removed, and the fibrous implant is placed. In yet another example, the existing structure stays in place, over/into/onto which the fibrous implant is placed.

In another embodiment, the fibrous implant can be used to occlude and/or close holes, ducts, or cavities.

In yet another embodiment, the fibrous implant can be used as a carrier containing either meshes, filters, coils, drugs, valves, strictures, or membranes.

One embodiment can be placed using conventional medical interventions either being invasive or minimally invasive procedures, for instance either by open surgery, MIDCAB, or an endovascular intervention. Such embodiment can be positioned in between body parts to act as an interposition, connect two body parts as a bypass or shunt, be positioned inside a body part as an endoluminal prosthesis, or be attached to or inserted into a body part.

### Implant Characteristics

The fibrous implant can be in the shape of, for instance, a mesh or tube with different varieties in their geometries, in one embodiment it is, for example either flat, curved, concave, convex, torqued, round, spherical and/or any combination thereof. The embodiment can comprise valves, meshes, filters, coils, local strictures or other additional components, which could be made out of fibers as well, by which those additional components could also induce elastin formation.

In some embodiments, the fibrous implant may have additional characteristics, such as or a combination of: structural support capacity, mechanical support capacity, expandability, self-expandability, crimp-ability, adhesive capacity, shape memory, swelling capacity, shrinking capacity, stiffening capacity, or be reactive to changes in for example temperature or acidity or to external stimuli such as for example ultrasound, magnetic fields, and or radiation by for instance light, heat, or sound.

In some embodiments, the fibrous implant may be used in combination with other implantable devices. The other implantable device will maintain its function, whereas the addition of the fibrous implant would enable elastin formation. There, the fibrous implant could be incorporated, fixed, adhered, coated, wrapped or mounted in any other way to the additional implantable device. The additional implantable device could for instance enable deliverability, provide mechanical support, ease flexibility, prevent kinking, enable fixation into a body part, occlude and/or close a body part such as vessels, ducts, holes, side branches, appendices, fistulas, and cavities. Those additional implantable devices could be for example stents, scaffolds, (bypass)grafts, valves, (balloon)catheters, closure devices, occluding devices, embolization devices, wires, coils, patches, covers, tubes, meshes, sheets endo or exo-prosthesis.

### Implantable Devices

Fibrous implants that enable elastin formation can shape different medical devices. Here, elastin formation could inhibit or prevent disease progression, and/or improve the mechanical properties of the tissue or implant. Such implants could be a single mesh to act for instance as a patch or have a tubular shape to act for instance as a stent, scaffold, (bypass)graft or shunt, but also could be a prosthesis such as a heart valve, or act as an occluding device.

### Example 1: Patch

In one embodiment, the fibrous implant is a mesh to act as a patch. In another embodiment, the patch can be made into any particular shape either predefined during manufacturing, or in yet another embodiment, tailor made from a sheet by the clinician. When placed onto an organ, elastin is ideally formed on the side facing the inside. The patch can either be mounted by sutures or staples, applied using an adhesive component, or can be self-adhesive.

### Example 2: Stents and Scaffolds

In another embodiment, the fibrous implants could be tubular shaped and be deployed inside the vasculature. These structural support devices are known as stents or scaffolds. When such devices are composed of a fibrous network, those scaffolds could be conceived as covered stents or stent grafts. Here, elastin is ideally formed on the endoluminal side of the implant. The structural support device could be made entirely out of fibers or is mounted onto/into an additional support device.

### Example 3: (Bypass) Grafts and Shunts

In yet another embodiment, the fibrous implants could be tubular shaped, where each end could be connected to a vessel to act as a bypass graft or fistula, be placed in-between an existing vessel to act as an interposition graft or be used to connect different vessels or body parts to act as a shunt. Elastin is here ideally formed on the endoluminal side of the implant. These implants are mostly placed surgically.

### Example 4: Valves

In yet another embodiment, the tubular fibrous implant comprises a valve with either one, two, three, or more leaflets. The valve could be as well entirely composed of a fibrous mesh, or such a fibrous mesh may be used to cover at least partially the valvular component. Here, elastin is ideally formed on the inflow side of the valve.

### Example 5: Embolization and Occluding Device

In yet another embodiment, the tubular fibrous implant comprises either a membrane, embolization component, or a swelling component, for which it could be used to occlude or embolize body parts. They can be implanted inside a vessel, by which the fibrous implant obstructs the blood flow. Also they can be placed inside a hole to prevent blood passage, and be used for instance to close septal defects or close an appendix. The occluding device could be made entirely out of fibers or be mounted onto/into and additional support device.

### Clinical Applications

The fibrous implant can be used in different applications where the formation of elastin has clinical relevance. In the cardiovascular system, elastin is present in cardiovascular vessels and valves, but also in the septum, atria, and myocardium of the heart.

### Endovascular Intervention

For treatment of vascular occlusion, several endovascular techniques are used to reestablish the blood flow. Each technique has the ability to induce damage to the native vascular tissue which could result in disease progression. To prevent reocclusion, the fibrous implant could be positioned into the vascular lumen after treatment. This could be done by endovascular implantation either using self-expandability or balloon expansion of the fibrous implant either with or without an additional support device.

### Example 1: Balloon Angioplasty

In one embodiment, the fibrous implant is used in combination with a delivery balloon. Plane balloon angioplasty is used to push away the vascular occlusion. Hereby, damage to the vascular wall can occur resulting in neointimal hyperplasia, which could be prevented by restoring the endoluminal elastin layer. In this way, a fibrous implant could be mounted onto the balloon of a balloon catheter which allows for implantation. Upon delivery the balloon inflates the fibrous implant to stay in place inside the vessel.

### Example 2: Percutaneous Intervention

In another embodiment, the fibrous implant can be used as a stent which can for instance be used to open vascular occlusions, reconstruct aneurysms, close of side branches, or comprise other implantable devices to be placed endovascularly. Here, the stent is either mounted on a balloon catheter or provides self-expandability, by which the stent/scaffold can be positioned and intraluminal prosthesis to keep the target vessel open. Common locations in which such a stent can be positioned are either cardiovascular such as in coronary arteries or in peripheral arteries such as in the upper and lower leg or carotid artery; to treat reocclusion in bypass grafts; or neurovascular such as in the brain.

### Example 3: Atherectomy

One embodiment is used for treatment after atherectomy. Atherectomy is used to remove vascular occlusion. There are several atherectomy techniques possible such as, directional, orbital, or rotational. These procedures will damage the endoluminal layer, by which disease progression can occur which could be prevented by restoring the endoluminal elastin layer.

### Example 4: Subintimal Bypass

In case the vessel is so occluded that true lumen passage is not feasible, a new passage can be created using subintimal bypass techniques. In this way, the subintimal layer of the occluded vessel is penetrated over which a medical device is advanced trough the subintimal space. After crossing the lesion, the medical device reenters the true lumen by which a new blood passage has been created. This technique can be used for treatment of chronic total occlusions either in the heart or peripheral vessels. This intervention however damages the subintimal layer which is prone to reocclusion. Here, one embodiment is used to cover the subintimal lumen to restore an endoluminal elastin layer.

### Example 5: Aneurysm Repair

For treatment of aneurysms, endovascular prostheses are used to create a new passage for blood inside the existing dilated vessel and prevent internal bleeding. These devices lack sufficient compliance and may result in vascular stiffening which could have a downstream effect leading to heart failure related to ventricular thickening or dilatation. Fibrous implants that could restore elastin could repair vascular elasticity and maintain vascular compliance. As such, one embodiment is used as an endoprosthesis for the treatment of aneurysm applications.

### Example 6: Closure, Embolization and Occlusion

In some embodiments, the fibrous implant may be used to close vessels or holes in tissues. For instance, in case a fistula has to be closed, or blood supply to a cancer has to be obstructed. Also, they can be used to close holes in the septum of the atrium or ventricle. Also, they can be used in the closure of patent ductus arteriosus in which endovascular plugs are used to close blood passages. They can be positioned inside a cavity to prevent debris from entering the blood stream in for instance atrium appendix applications.

### Example 7: Valvular Replacement

One embodiment comprising a valve may be used to treat patients suffering from congenital defects such as Epstein Anomaly, atresia, or pulmonary valve stenosis. Also it can happen that valve leaflets did not split properly or lack the ability to close entirely. Even so valvular diseases could be acquired resulting is valvular calcification causing stenosis and regurgitation. As a minimally invasive alternative to endovascular valve positioning, valve prostheses may be placed using a transapical approach. Valves may also be placed in other locations rather than the heart, such as for treatment of patients suffering from deep vein thrombosis, in order to replace the venous valve.

### Surgical Intervention

As an alternative to an endovascular approach, surgical interventions might still be needed to overcome acquired or congenital defects. Sometimes affected tissue is either first removed and replaced by a fibrous implant, left in place and repaired, or left in place and replaced.

### Example 8: Patching

In one embodiment, the fibrous implant is a patch that can be used for wound healing, to close holes in the vasculature from either congenital or acquired defects such as a disease or accident, or for surgical reconstruction. This might for instance be useful in case vascular branches are separated to repair the open hole in the vascular wall. In case of aneurysm indications, it could function to reinforce the vascular wall from the outside. Also it may be used to repair organs such as the heart, where these fibrous patches could for instance be applied onto the heart to restore the myocardium or by placing a patch inside the heart to for instance close septal defects and repair individual heart valve leaflets.

In one example, surgeons could adhere fibrous patches by for instance, gluing, suturing, stapling, self-adhering, heating or any other external stimuli to mount the patch. The patch can be mounted onto surfaces of organs such as the heart, but also be used to repair tubular shapes and could also be used to repair or at least partially replace damaged valves.

### Example 9: Bypassing and Interpositioning

In another embodiment, surgeons use tubular structures to replace or repair vascular constructs. Here, an embodiment acting as a bypass graft could be either sutured to the existing vasculature by at least one end of the anastomosis or contain at least one structural support device at the ends, such as a stent or scaffold, to be positioned inside the existing vasculature. Another embodiment acts as an interposition graft to replace for instance parts of the carotid artery or abdominal aorta. Yet another embodiment acts as a shunt to treat for instance acquired or congenital heart defects, act as an access grafts for kidney dialyses, or for cerebral, pulmonary, or portosystemic indications.

### Example 10: Valvular Repair or Replacement

In yet another example, the embodiment is a tubular conduit and comprises valves. This embodiment can be surgically implanted to replace the existing valve. This conduit could have different shapes and include for instance a sinus shape, and account for the inflow access to the coronary arteries in case of an aortic valve replacement. In yet another embodiment, tubular conduits can comprise single or multiple valves in series to act as a interposition or bypass graft for vein indications in for instance the leg.

### Example 11: Pediatric Applications

The embodiment is in particular of use in pediatric applications. Here, young patients who will still undergo somatic growth and suffering from either congenital or acquired cardiovascular diseases or defects will benefit from an implant that can restore elastin to regain functionality such as vasomotion in case of use in vascular applications. In addition, the potential added benefit of using a biodegradable embodiment in these cases will even provide additional benefits by accommodating somatic growth.

### Methods of Producing

In an exemplary embodiment, implants can be made by production techniques in which fibers can be obtained, preferably by electrospinning. Other technologies in which fibrous implants could be made can be a variation of electrospinning such as melt, wet, emulsion, coaxial, stable jet and near field electrospinning, but also other fiber producing technologies such as 3D printing, electrostatic drawing, braiding, weaving, knitting, additive manufacturing, bioprinting, electro spraying, polymer jetting, injection molding, casting or any combination thereof.

When using electrospinning, fiber diameter, fiber orientation and pore size can be tuned by applying distinct combinations of settings such as voltage, rotation speed, spinning distance, coaxial flow, polymer inflow speed, target size, target needle diameter, and ambient settings such as humidity and temperature. Also, multiple spinning nozzles can be used simultaneously where each nozzle is spinning different fiber configurations.

In one variation, the embodiment is at least partially composed of distinct layers. A fibrous network could be obtained by collecting multiple fibers on top of each other, forming layers of fibers. By altering the production settings, the layers could have different densities, fiber diameters or pore size. Also, the number of layers will define the thickness of the implant. In this way, the inner surface of a tubular conduit could for instance have a different layer of fibers than the outer surface of the tube.

In one other variation, the embodiment is at least partially composed of different materials. The network could be composed out of fibers from the same material, or of fibers with different materials. Also, layers could be made out of distinct fibers composed of different materials. In this way, the inner surface of a tubular conduit could for instance have a different degradation speed compared to the outer surface.

In yet another variation, the embodiment is at least partially composed of fibers comprising different materials. Also, individual fibers might be composed out of distinct material compositions by either coaxial or multi-axial spinning in which individual fibers can be composed of a different core and outer layer or multiple layers.

Embodiments using electrospinning could be made into sheets by spinning on a flat collector, or tubular shape by using tubular collectors, which either rotate themselves, or around which the nozzles rotate. Here, collectors could have any imaginable shape on which a fibrous implant could be produced.

In a variation, the embodiment can be at least partially self-adhesive or include an adhesive such as a glue or biological adhesive component, either at one side or multiple sides of the implant and/or inside the implant.

Some embodiments could be incorporated with additional implantable devices by either adhering, suturing, crimping, stapling, or physically enclosing by for instance sandwiching.

In another variation of an embodiment, the fibrous implant is directly applied on an additional support or delivery device using for instance electrospinning, which could be provided with a fibrous network on any part of such implant, being on a surface, inside or attached to the additional implantable device. In this way, fibrous implants could be incorporated with additional implantable devices, such as balloon catheters, stents, grafts, valves, or occluding devices.

In one exemplary embodiment, the additional delivery device is a stent provided with a fibrous implant on the inner side of the stent facing the lumen, and/or on the outer side of the stent facing the native wall. In another variation the embodiment is sandwiched between multiple stents

In yet another exemplary embodiment, the additional delivery device is a balloon catheter which is provided with a fibrous implant by applying it directly onto the balloon in either an inflated or deflated state.

In yet another exemplary embodiment, occluding devices could be provided with a fibrous implant on the inner side, outer side or in between the implant structure or a combination thereof.

In yet another example embodiment, the clinician could spray the fibrous network directly on the targeted tissue or organs in need of elastin formation.

### Methods of Testing

Fibrous implants can be partially characterized using scanning electron microscopy (SEM). Using high magnifications, (averaged) fiber diameter, and (averaged) pore size can be defined. Lower SEM magnifications may be used to characterize the network organization, in which post processing using image analyses software such as ImageJ could be used to determine the network organization and level of fiber alignment.

Crystallinity of the polymer fibers before, during and after production may be determined using Differential Scanning calorimetry (DSC). Molecular weight of the polymer may be determined using Gel Permeation Chromatography (GPC).

The ability of fibrous implants to induce elastin formation can be tested in animals, such as rats, or even more preferably in more translational animal models such as rabbits or pigs.

Implants can be assessed for their ability to induce elastin by histology after explanation. This can be achieved by using for instance a Verhoeff's staining, or a combined staining including a Verhoeff's staining such as Russell-Moval Pentachrome staining or using an immunofluorescent staining using a primary antibody specific to elastin. This can be at predefined time-points to evaluate cell migration and tissue formation over time.

### Example 1: The Effect of Elastin Formation Using Intraluminal Prosthesis with Different Fiber Diameters

Two distinct embodiments were evaluated for their capacity to induce elastin formation. One embodiment was a tubular intraluminal implant composed of small fibers. The second embodiment was also a tubular intraluminal implant, composed of two distinct layers having large fibers on the outside, and small fibers on the inside. The influence of fiber thickness on elastin formation was evaluated in rabbits. Both fibrous implants where positioned in the femoral artery of rabbits as an intraluminal prosthesis using a balloon catheter. After 12 weeks, the implants where explanted and evaluated for the presence of elastin using Russell-Moval Pentachrome staining. Prior to implantation, fibrous implants where characterized using SEM.

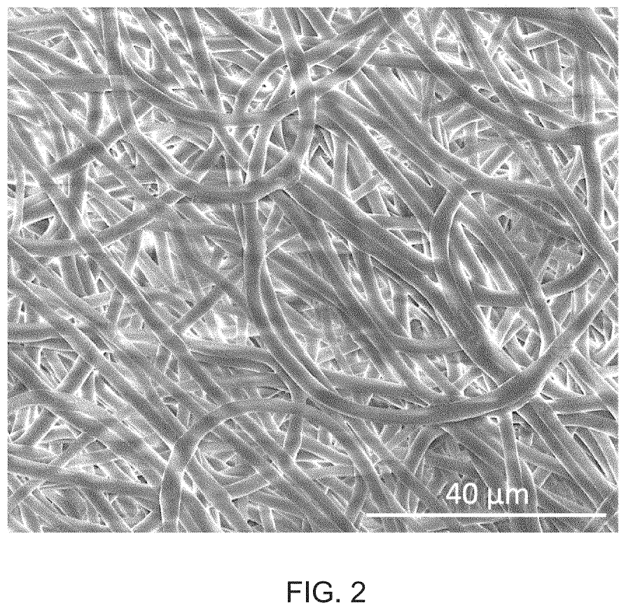

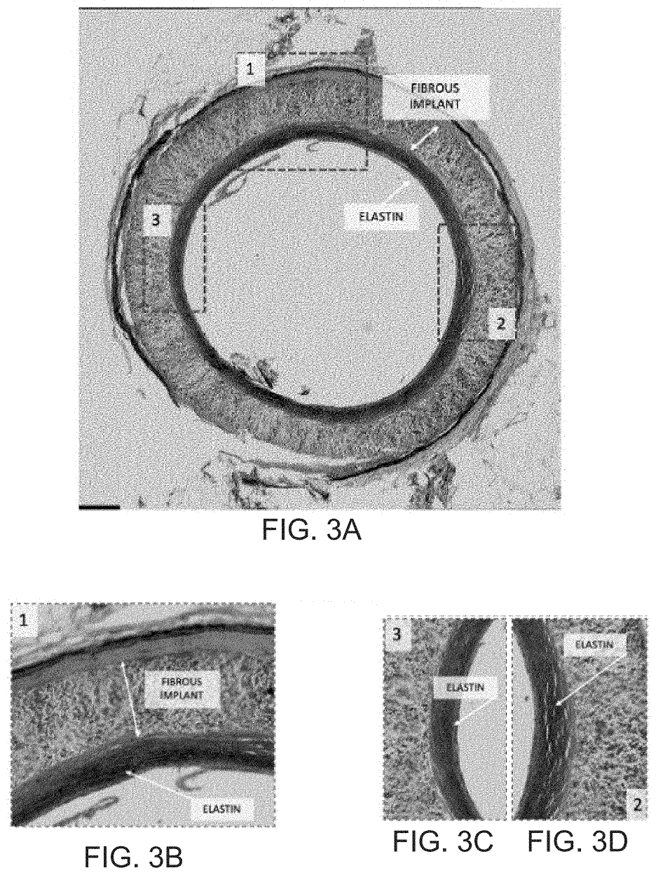

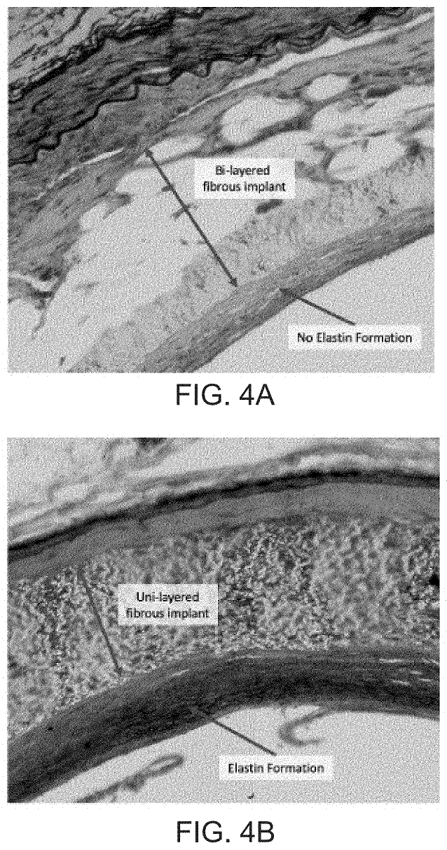

An exemplary embodiment composed of small fibers was characterized using SEM prior to implantation. The luminal side after balloon catheter expansion revealed the following network morphology as indicated in FIG. 2. This 3-month explant revealed abundant endoluminal elastin formation as indicated in FIGS. 3A-D. Compared to the other exemplary embodiment with an additional outer layer composed of larger fibers, after 3 months of implantation, this dual layer configuration did not induce elastin formation (FIG. 4A) to the extent that was observed in the embodiment comprised only out of small fibers (FIG. 4B). As both embodiments where composed of the same material, and the inner layer of the of the dual layered implant had the same fibrous network morphology as the single layer implant, this might suggest that fiber size can be used to steer elastin formation.

### Example 2: The Influence of α-Sma Positive Cells on Elastin Formation

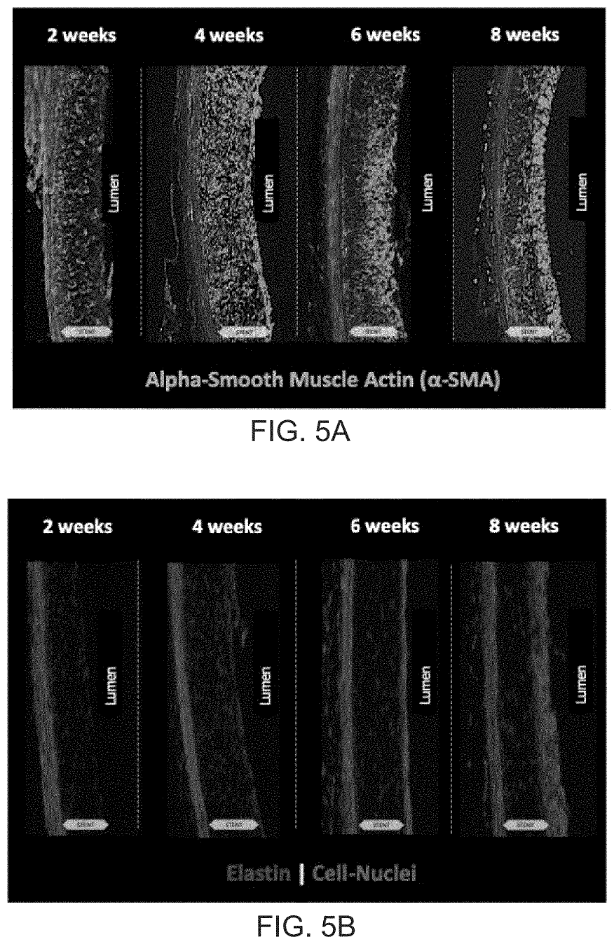

To investigate the origin of elastin production in-situ, several comparable embodiments have been positioned as endoluminal prosthesis in the abdominal aorta of rats for 2, 4, 6, and 8 weeks, using a balloon catheter. These embodiments where solely composed of small fibers, comparable to the embodiment as described in Example 1 Immunofluorescent staining was applied for alpha-smooth muscle actin (α-SMA) as shown in FIG. 5A and elastin as shown in FIG. 5B. Gradual infiltration of α-SMA positive cells can be seen from the adventitia toward the luminal side of the implant. Over time, endoluminal elastin formation was observed, which at 6 and 8 weeks colocalized with the presence of alpha-smooth muscle actin positive cells.

This might suggest that cells expressing alpha-smooth muscle actin might be involved in the formation of elastin. Since the elastin is formed on the luminal side of the implant, and not inside the fibrous network, the proximity to the blood stream could have a positive effect on elastin formation.

1. A cardiovascular fibrous implant for rebuilding elastin,
wherein the implant is comprised of fibers forming a network, and
wherein the fibers comprised in said network have a fiber diameter of 150 μm or less.

2. The cardiovascular fibrous implant according to claim 1, wherein said fibrous implant is at least partially bioabsorbable.

3. The cardiovascular fibrous implant according to claim 1, wherein said fibrous implant comprises a drug and/or adhesive component.

4. The cardiovascular fibrous implant according to claim 1, wherein the fibers are coated with a coating layer and wherein the coating layer comprises a drug and/or adhesive component.

5. The cardiovascular fibrous implant according to claim 1, wherein said fibrous implant comprises fibers and/or pores that allow cells to adhere, migrate, infiltrate, proliferate, (trans)differentiate, synthesise tissue and combinations thereof.

6. The cardiovascular fibrous implant according to claim 1, wherein said fibrous implant is composed of multiple layers of fibers, wherein the multiple layers of fibers comprise at least a first layer of fibers and a second layer of fibers, and wherein the first layer of fibers is different from the second layer of fibers.

7. The cardiovascular fibrous implant according to claim 6, wherein the first layer of fibers is different from the second layer of fibers in parameters selected from the group consisting of fiber diameter, pore size, density, stiffness, fiber organization, material composition, drug composition, adhesive components, degradation speed, and combinations thereof.

8. The cardiovascular fibrous implant according to claim 1, wherein said fibrous implant comprises:
i) a planar shape;
ii) a tubular shape, wherein said tubular shape comprises:
a. a valve to be used as a valvular prosthesis;
b. either a membrane, embolization or swelling component to be used as a closure device, embolization device or occlusion device.

9. The cardiovascular fibrous implant according to claim 1, wherein said fibrous implant is capable of reconstructing at least partially a vascular media and/or intima layer by additionally rebuilding a smooth muscle layer and/or endothelium layer.

10. The cardiovascular fibrous implant according to claim 8, wherein the planar shaped fibrous implant is a patch.

11. The cardiovascular fibrous implant according to claim 8, wherein the tubular shaped fibrous implant is a scaffold, stent, covered stent, stent graft, graft, bypass graft, fistula or shunt.

12. A method of rebuilding extracellular matrix (ECM) components in a host comprising:
implanting into the host a fibrous implant comprising fibers forming a network and having a fiber diameter of 150 μm or less, wherein the fibrous implant is at least partially:
i) brought into direct contact with blood flow; and/or
ii) brought into direct contact with native tissue, wherein said native tissue is selected from the group consisting of healthy native tissue, injured native tissue, damaged native tissue, diseased native tissue and combinations thereof.

13. The method according to claim 12, wherein the fibrous implant is attached to an additional support or delivery device.

14. The method according to claim 12, wherein the fibrous implant is exposed to mechanical loading conditions, wherein said mechanical loading conditions are either static mechanical loading conditions and/or dynamic mechanical loading conditions, and wherein said mechanical loading conditions are selected from the group consisting of shear stress, pre-stress, pre-strain, residual stress, stretch, compression and combinations thereof.

15. (canceled)
